(19) United States
(12) Patent Application Publication
(10) Pub. No.: US 2024/0331809 A1
(43) Pub. Date: Oct. 3, 2024

(54) Artificial Intelligence Systems and Processes for In Silico Discovery of Immune Modulators and T Regulatory Cell Screening Methodologies

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Timothy Warbington, Phoenix, AZ (US); Courtney Bartlett, Niceville, FL (US)

(73) Assignee: CREATIVE MEDICAL TECHNOLOGIES, INC., Phoenix, AZ (US)

(21) Appl. No.: 18/623,197

(22) Filed: Apr. 1, 2024

Related U.S. Application Data
(60) Provisional application No. 63/455,965, filed on Mar. 30, 2023.

(51) Int. Cl. G16C 20/10; G01N 33/50; G16B 20/50; G16C 20/70

(52) U.S. Cl. CPC G16C 20/10; G01N 33/5055; G16B 20/50; G16C 20/70; G01N 2333/4716

(57) ABSTRACT

Disclosed are methods, means and systems of identifying compounds and augment T regulatory cell activity and/or number in vitro and/or in vivo utilizing deep learning approaches. Systems for screening compound libraries in silico are provided as well as laboratory methods of testing modulation of T regulatory cell activity and/or numbers. Results provided by the disclosure will serve as the basis for increasing T regulatory cells, which is desirable in situations of autoimmunity or organ transplantation. In situations of oncology or infectious disease reduction of T regulatory cell number and/or activity is desired.

### CROSS-REFERENCE TO RELATED APPLICATIONS

The present application claims benefit of Canadian Patent Application Serial No. 63/455,965, filed on Mar. 30, 2023, entitled Artificial Intelligence Systems and Processes for In Silico Discovery of Immune Modulators and T Regulatory Cell Screening Methodologies, the contents of which are incorporated herein by reference in its entirety.

### FIELD OF THE INVENTION

The teachings herein relate to the use of artificial intelligence systems for discovery of immune modulators and T regulatory cell screenings.

### BACKGROUND OF THE INVENTION

Biological knowledge is accumulating at an accelerating pace. This geometric expansion of knowledge has been catalyzed by several significant discoveries including: a) the introduction of DNA chips, which allow for mass read out of biological systems; b) the microarray/bioinformatics technologies, which allow for rapid integration of existing biological pathways and determine which conserved molecular cascades are being activated in specific contexts; and c) the storage and public sharing of raw data allowing for individuals or systems to mine existing information resources.

Due to the incredible rise in available date, the ability of the human mind to process develop meaningful experimental hypothesis is increasing in difficulty.

The concept of Fuzzy Set and Fuzzy Theory was introduced by Lotfi A. Zadeh in his famous paper, in 1965 (as a professor of University of California, at Berkeley). Since then, many people have worked on the Fuzzy Logic technology and science. Fuzzy logic is a mathematical theory that deals with reasoning and decision-making in situations where there is uncertainty, imprecision, or ambiguity. Unlike traditional logic, which deals with binary true/false values, fuzzy logic allows for degrees of truth or membership to be assigned to statements or propositions. Fuzzy logic is based on the idea that many real-world problems cannot be expressed in terms of precise, binary values, and instead require a more nuanced approach. For example, in a temperature control system, it may not be enough to simply turn a heater on or off based on a set temperature threshold, as the temperature may fluctuate around that threshold. Fuzzy logic allows for a more gradual response, with the degree of heating or cooling varying based on the degree of membership of the current temperature to different fuzzy sets.

There have been previous attempts to utilize fuzzy logic to identify drug-target interactions. For example, Yu et al (Mol Omics. 2020 Dec. 1;16 (6): 583-591) developed a new fusion method, called FPSC-DTI, that fuses feature projection fuzzy classification (FP) and super cluster classification (SC) to predict DTI. As the experimental result, the mean percentile ranking (MPR) that was yielded by FPSC-DTI achieved 0.043, 0.084, 0.072, and 0.146 on enzyme, ion channel (IC), G-protein-coupled receptor (GPCR), and nuclear receptor (NR) datasets, respectively. And the AUC values exceeded 0.969 over all four datasets.

The application of fuzzy logic principles as well as artificial intelligence-based data analysis has not been utilized in identification of agents to modulate immunological cell activity.

### SUMMARY OF THE INVENTION

Preferred methods include embodiments of identifying agents capable of modulating T regulatory cell activity comprising the steps of: a) identifying a list of compounds possessing pharmacologically acceptable properties for therapeutic use; b) utilizing an artificial intelligence means to assess ability of said compounds from “a” to modulate ability of FoxP3 protein to interact with target sites on DNA; c) enhancing ability of said compounds identified from step “b” to modulate FoxP3 DNA binding by performing chemical optimization steps; and d) assessing activity of said identified compounds using in vitro T regulatory cell assessment assays.

Preferred embodiments include methods wherein said T regulatory cell activity is modulation of macrophage activity.

Preferred embodiments include methods wherein said macrophages are plastic adherent cells.

Preferred embodiments include methods wherein said macrophages are capable of opsonization.

Preferred embodiments include methods wherein said macrophages are capable of antibody dependent cytotoxicity.

Preferred embodiments include methods wherein said macrophages are capable of generating TNF-alpha after TLR4 activation.

Preferred embodiments include methods wherein said TLR4 agonist is lipopolysaccharide.

Preferred embodiments include methods wherein said TLR4 agonist is beta glucan.

Preferred embodiments include methods wherein said TLR4 agonist is HMGB1.

Preferred embodiments include methods wherein said TLR4 agonist is lipoteichoic acid.

Preferred embodiments include methods wherein said TLR4 agonist is monophosphoryl lipid A.

Preferred embodiments include methods wherein said TLR4 agonist is paclitaxel.

Preferred embodiments include methods wherein said TLR4 agonist is Bacillus Calmette-Guerin.

Preferred embodiments include methods wherein said TLR4 agonist is hyaluronic acid.

Preferred embodiments include methods wherein said hyaluronic acid is low molecular weight hyaluronic acid.

Preferred embodiments include methods wherein said low molecular weight hyaluronic acid possesses a molecular weight of 10-1000 kDA.

Preferred embodiments include methods wherein said low molecular weight hyaluronic acid possesses a molecular weight of 10-500 kDA.

Preferred embodiments include methods wherein said low molecular weight hyaluronic acid possesses a molecular weight of 10-50 kDA.

Preferred embodiments include methods wherein said TLR4 agonist is glucopyranosyl Lipid A (GLA).

Preferred embodiments include methods wherein said TLR4 agonist is oxidized low-density lipoprotein (oxLDL).

Preferred embodiments include methods wherein said TLR4 agonist is resiquimod.

Preferred embodiments include methods wherein said TLR4 agonist is Pam3CSK4

Preferred embodiments include methods wherein said macrophages express CD16.

Preferred embodiments include methods wherein said macrophages express CD14.

Preferred embodiments include methods wherein said macrophages express CDD68.

Preferred embodiments include methods wherein said macrophages express interleukin-1 beta receptor.

Preferred embodiments include methods wherein said macrophages express interleukin-4 receptor.

Preferred embodiments include methods wherein said macrophages express interleukin-13 beta receptor.

Preferred embodiments include methods wherein said macrophages express interferon gamma receptor.

Preferred embodiments include methods wherein said macrophages express HGF-1 receptor.

Preferred embodiments include methods wherein said macrophages LRP-1.

Preferred embodiments include methods wherein said macrophages express HDL receptor.

Preferred embodiments include methods wherein said macrophages express HLA-I.

Preferred embodiments include methods wherein said macrophages increase expression of HLA-I upon treatment with interferon gamma.

Preferred embodiments include methods wherein said macrophages increase expression of HLA-I upon treatment with an activator of NF-kappa B.

Preferred embodiments include methods wherein said activator of NF-kappa B is necrotic tissue.

Preferred embodiments include methods wherein said activator of NF-kappa B is TNF-alpha.

Preferred embodiments include methods wherein said activator of NF-kappa B is lymphotoxin.

Preferred embodiments include methods wherein said activator of NF-kappa B is interferon alpha.

Preferred embodiments include methods wherein said activator of NF-kappa B is interferon beta.

Preferred embodiments include methods wherein said activator of NF-kappa B is interferon gamma.

Preferred embodiments include methods wherein said activator of NF-kappa B is interferon tau.

Preferred embodiments include methods wherein said activator of NF-kappa B is interleukin-8.

Preferred embodiments include methods wherein said activator of NF-kappa B is interleukin-12.

Preferred embodiments include methods wherein said activator of NF-kappa B is interleukin-15.

Preferred embodiments include methods wherein said activator of NF-kappa B is interleukin-17.

Preferred embodiments include methods wherein said activator of NF-kappa B is interleukin-17C.

Preferred embodiments include methods wherein said activator of NF-kappa B is interleukin-17F.

Preferred embodiments include methods wherein said activator of NF-kappa B is conditioned media from interleukin 17 treated neutrophils.

Preferred embodiments include methods wherein said activator of NF-kappa B is conditioned media from interleukin 17 treated mesenchymal stem cells.

Preferred embodiments include methods wherein said mesenchymal stem cells express CD90.

Preferred embodiments include methods wherein said mesenchymal stem cells express CD105.

Preferred embodiments include methods wherein said mesenchymal stem cells express c-kit.

Preferred embodiments include methods wherein said mesenchymal stem cells express E-cadherin.

Preferred embodiments include methods wherein said mesenchymal stem cells express VLA4.

Preferred embodiments include methods wherein said mesenchymal stem cells express hTERT.

Preferred embodiments include methods wherein said mesenchymal stem cells express Brother of the Regulator of Imprinted Sites.

Preferred embodiments include methods wherein said mesenchymal stem cells express extracellular endosomal associated protein.

Preferred embodiments include methods wherein said mesenchymal stem cells express HLA-E.

Preferred embodiments include methods wherein said mesenchymal stem cells express HLA-G.

Preferred embodiments include methods wherein said activator of NF-kappa B is conditioned media from interleukin 6 treated endothelial cells.

Preferred embodiments include methods wherein endothelial cells are derived from hematopoietic stem cells.

Preferred embodiments include methods wherein said hematopoietic stem cells express CD133.

Preferred embodiments include methods wherein said hematopoietic stem cells express CD34.

Preferred embodiments include methods wherein said hematopoietic stem cells express c-met.

Preferred embodiments include methods wherein said hematopoietic stem cells express interleukin-3 receptor.

Preferred embodiments include methods wherein said hematopoietic stem cells express interleukin-6 receptor.

Preferred embodiments include methods wherein said hematopoietic stem cells express interleukin-11 receptor.

Preferred embodiments include methods wherein said hematopoietic stem cells express steel factor receptor.

Preferred embodiments include methods wherein said hematopoietic stem cells express interferon alpha receptor.

Preferred embodiments include methods wherein said hematopoietic stem cells are differentiated to endothelial cells through treatment with leukemia inhibitor factor, and/or vascular endothelial growth factor, and/or bone morphogenic 2.

Preferred embodiments include methods wherein said activator of NF-kappa B is conditioned media from interleukin 6 treated dendritic cells.

Preferred embodiments include methods wherein said activator of NF-kappa B is conditioned media from interleukin 6 treated natural killer cells.

Preferred embodiments include methods wherein said activator of NF-kappa B is conditioned media from interleukin 6 treated type 1 B cells.

Preferred embodiments include methods wherein said activator of NF-kappa B is conditioned media from interleukin 6 treated natural killer T cells.

Preferred embodiments include methods wherein said activator of NF-kappa B is conditioned media from interleukin 6 treated gamma delta T cells.

Preferred embodiments include methods wherein said T regulatory cells are assessed for ability to suppress interferon gamma induced maturation of M1 macrophages.

Preferred embodiments include methods wherein said T regulatory cells are assessed for ability to suppress interleukin-1 beta induced maturation of M1 macrophages.

Preferred embodiments include methods wherein said T regulatory cells are assessed for ability to suppress tumor necrosis factor alpha induced maturation of M1 macrophages.

Preferred embodiments include methods wherein said T regulatory cells are assessed for ability to suppress lymphotoxin induced maturation of M1 macrophages.

Preferred embodiments include methods wherein said T regulatory cells are assessed for ability to suppress beta glucan induced maturation of M1 macrophages.

Preferred embodiments include methods wherein said T regulatory cells are assessed for ability to suppress cytotax induced maturation of M1 macrophages.

Preferred embodiments include methods wherein said T regulatory cells are assessed for ability to suppress HMBG1 induced maturation of M1 macrophages.

Preferred embodiments include methods wherein said T regulatory cells are assessed for ability to suppress alpha galactosylceramide induced maturation of M1 macrophages.

Preferred embodiments include methods wherein said T regulatory cells are assessed for ability to suppress Th1 cell induced maturation of M1 macrophages.

Preferred embodiments include methods wherein said T regulatory cells are assessed for ability to suppress anti-CD3, anti-CD28 activated T cell induced maturation of M1 macrophages.

Preferred embodiments include methods wherein said T regulatory cells are assessed for ability to suppress phytohemagglutinin activated T cell induced maturation of M1 macrophages.

Preferred embodiments include methods wherein said T regulatory cells are assessed for ability to suppress pokeweed mitogen activated T cell induced maturation of M1 macrophages.

Preferred embodiments include methods wherein said T regulatory cells are assessed for ability to suppress allogeneic mixed lymphocyte reaction activated T cell induced maturation of M1 macrophages.

Preferred embodiments include methods wherein said T regulatory cells are assessed for ability to suppress poly IC induced maturation of M1 macrophages.

Preferred embodiments include methods wherein said T regulatory cells are assessed for ability to suppress CpG DNA induced maturation of M1 macrophages.

Preferred embodiments include methods wherein said T regulatory cells are assessed for ability to suppress BCG induced maturation of M1 macrophages.

Preferred embodiments include methods wherein said T regulatory cells are assessed for ability to suppress complement C3 induced maturation of M1 macrophages.

Preferred embodiments include methods wherein said T regulatory cells are assessed for ability to suppress complement C5 induced maturation of M1 macrophages.

Preferred embodiments include methods wherein said T regulatory cells are assessed for ability to suppress neutrophil extracellular trap induced maturation of M1 macrophages.

Preferred embodiments include methods wherein said maturation of M1 macrophages is increased phagocytic activity.

Preferred embodiments include methods wherein said phagocytic activity is quantified by ingestion of zymosan.

Preferred embodiments include methods wherein said phagocytic activity is quantified by ingestion of inactivated bacteria.

Preferred embodiments include methods wherein said phagocytic activity is quantified by ingestion of mycoplasma tuberculosis bacteria.

Preferred embodiments include methods wherein said maturation of M1 macrophages is increased expression of CD40 as compared to a macrophage in an immature state.

Preferred embodiments include methods wherein said maturation of M1 macrophages is increased expression of CD80 as compared to a macrophage in an immature state.

Preferred embodiments include methods wherein said maturation of M1 macrophages is increased expression of CD80 as compared to a macrophage in an immature state.

Preferred embodiments include methods wherein said maturation of M1 macrophages is increased expression of CD86 as compared to a macrophage in an immature state.

Preferred embodiments include methods wherein said maturation of M1 macrophages is increased production of interleukin-12 as compared to a macrophage in an immature state.

Preferred embodiments include methods wherein said maturation of M1 macrophages is increased expression of nitric oxide as compared to a macrophage in an immature state.

Preferred embodiments include methods wherein said maturation of M1 macrophages is increased expression of interleukin-2 as compared to a macrophage in an immature state.

Preferred embodiments include methods wherein said maturation of M1 macrophages is increased expression of interleukin-6 as compared to a macrophage in an immature state.

Preferred embodiments include methods wherein said maturation of M1 macrophages is increased expression of macrophage migration inhibitory protein alpha as compared to a macrophage in an immature state.

Preferred embodiments include methods wherein said maturation of M1 macrophages is increased expression of macrophage migration inhibitory protein beta as compared to a macrophage in an immature state.

Preferred embodiments include methods wherein said maturation of M1 macrophages is increased expression of macrophage activation factor as compared to a macrophage in an immature state.

Preferred embodiments include methods wherein said maturation of M1 macrophages is increased expression of matrix metalloprotease 1 as compared to a macrophage in an immature state.

Preferred embodiments include methods wherein said maturation of M1 macrophages is increased expression of matrix metalloprotease 3 as compared to a macrophage in an immature state.

Preferred embodiments include methods wherein said maturation of M1 macrophages is increased expression of matrix metalloprotease 5 as compared to a macrophage in an immature state.

Preferred embodiments include methods wherein said maturation of M1 macrophages is increased expression of matrix metalloprotease 9 as compared to a macrophage in an immature state.

Preferred embodiments include methods wherein said maturation of M1 macrophages is increased expression of matrix metalloprotease 13 as compared to a macrophage in an immature state.

Preferred embodiments include methods wherein said maturation of M1 macrophages is increased expression of calpain 1 as compared to a macrophage in an immature state.

Preferred embodiments include methods wherein said maturation of M1 macrophages is increased expression of calpain 6 as compared to a macrophage in an immature state.

Preferred embodiments include methods wherein said maturation of M1 macrophages is increased expression of membrane ceramide as compared to a macrophage in an immature state.

Preferred embodiments include methods wherein said maturation of M1 macrophages is increased expression of TRAIL as compared to a macrophage in an immature state.

Preferred embodiments include methods wherein said maturation of M1 macrophages is increased expression of Fas ligand as compared to a macrophage in an immature state.

Preferred embodiments include methods wherein said maturation of M1 macrophages is increased expression of endostatin as compared to a macrophage in an immature state.

Preferred embodiments include methods wherein said maturation of M1 macrophages is increased expression of tissue inhibitor of metalloprotease-1 as compared to a macrophage in an immature state.

Preferred embodiments include methods wherein said maturation of M1 macrophages is increased expression of tissue inhibitor of metalloprotease-5 as compared to a macrophage in an immature state.

Preferred embodiments include methods wherein said maturation of M1 macrophages is increased expression of urokinase plasminogen activator as compared to a macrophage in an immature state.

Preferred embodiments include methods wherein said maturation of M1 macrophages is increased expression of superoxide radicals as compared to a macrophage in an immature state.

Preferred embodiments include methods wherein said maturation of M1 macrophages is increased expression of hydroxyl radicals as compared to a macrophage in an immature state.

Preferred embodiments include methods wherein said T regulatory cell modulation of macrophage activity is upregulation of activity of M2 macrophages.

Preferred embodiments include methods wherein said M2 macrophages preferentially express TGF-beta as compared to M1 macrophages.

Preferred embodiments include methods wherein said M2 macrophages preferentially express interleukin-4 as compared to M1 macrophages.

Preferred embodiments include methods wherein said M2 macrophages preferentially express arginase as compared to M1 macrophages.

Preferred embodiments include methods wherein said M2 macrophages are preferentially angiogenic as compared to M1 macrophages.

Preferred embodiments include methods wherein said preferential angiogenic activity of M2 macrophages is due to reduced production of nitric oxide as compared to M1 macrophages.

Preferred embodiments include methods wherein said preferential angiogenic activity of M2 macrophages is due to reduced production of interferon gamma as compared to M1 macrophages.

Preferred embodiments include methods wherein said preferential angiogenic activity of M2 macrophages is due to increased production of GDF-1 as compared to M1 macrophages.

Preferred embodiments include methods wherein said preferential angiogenic activity of M2 macrophages is due to increased production of GDF-5 as compared to M1 macrophages.

Preferred embodiments include methods wherein said preferential angiogenic activity of M2 macrophages is due to increased production of GDF-11 as compared to M1 macrophages.

Preferred embodiments include methods wherein said preferential angiogenic activity of M2 macrophages is due to increased production of angiogenin as compared to M1 macrophages.

Preferred embodiments include methods wherein said preferential angiogenic activity of M2 macrophages is due to increased production of angiopoietin-1 as compared to M1 macrophages.

Preferred embodiments include methods wherein said preferential angiogenic activity of M2 macrophages is due to increased production of Del-1 as compared to M1 macrophages.

Preferred embodiments include methods wherein said preferential angiogenic activity of M2 macrophages is due to increased production of FGF1 as compared to M1 macrophages.

Preferred embodiments include methods wherein said preferential angiogenic activity of M2 macrophages is due to increased production of FGF2 as compared to M1 macrophages.

Preferred embodiments include methods wherein said preferential angiogenic activity of M2 macrophages is due to increased production of FGF5 as compared to M1 macrophages.

Preferred embodiments include methods wherein said preferential angiogenic activity of M2 macrophages is due to increased production of follistatin as compared to M1 macrophages.

Preferred embodiments include methods wherein said preferential angiogenic activity of M2 macrophages is due to increased production of granulocyte colony stimulating factor as compared to M1 macrophages.

Preferred embodiments include methods wherein said preferential angiogenic activity of M2 macrophages is due to increased production of interleukin-8 as compared to M1 macrophages.

Preferred embodiments include methods wherein said preferential angiogenic activity of M2 macrophages is due to increased production of interleukin-20 as compared to M1 macrophages.

Preferred embodiments include methods wherein said preferential angiogenic activity of M2 macrophages is due to increased production of leptin as compared to M1 macrophages.

Preferred embodiments include methods wherein said preferential angiogenic activity of M2 macrophages is due to increased production of midkine as compared to M1 macrophages.

Preferred embodiments include methods wherein said preferential angiogenic activity of M2 macrophages is due to increased transcription of HIF-1 alpha as compared to M1 macrophages.

Preferred embodiments include methods wherein said preferential angiogenic activity of M2 macrophages is due to increased transcription of NF-kappa B as compared to M1 macrophages.

Preferred embodiments include methods wherein said preferential angiogenic activity of M2 macrophages is due to increased transcription of brother of the regulator of imprinted sites as compared to M1 macrophages.

Preferred embodiments include methods wherein said preferential angiogenic activity of M2 macrophages is due to increased production of placental growth factor as compared to M1 macrophages.

Preferred embodiments include methods wherein said preferential angiogenic activity of M2 macrophages is due to increased production of PDGF-BB as compared to M1 macrophages.

Preferred embodiments include methods wherein said preferential angiogenic activity of M2 macrophages is due to increased production of pleiotrophin as compared to M1 macrophages.

Preferred embodiments include methods wherein said preferential angiogenic activity of M2 macrophages is due to increased production of progranulin as compared to M1 macrophages.

Preferred embodiments include methods wherein said preferential angiogenic activity of M2 macrophages is due to increased production of proliferin as compared to M1 macrophages.

Preferred embodiments include methods wherein said T regulatory cell activity is modulation of effector T cell activity.

Preferred embodiments include methods wherein said effector T cell is a T helper cell.

Preferred embodiments include methods wherein said T helper cell expresses CD40 ligand.

Preferred embodiments include methods wherein said CD40 ligand expressed on said T helper cell is capable of inducing maturation of an antigen presenting cell.

Preferred embodiments include methods wherein said antigen presenting cell is an endothelial cell.

Preferred embodiments include methods wherein said antigen presenting cell is a neutrophil.

Preferred embodiments include methods wherein said antigen presenting cell is a dendritic cell.

Preferred embodiments include methods wherein said antigen presenting cell is a B cell.

Preferred embodiments include methods wherein said antigen presenting cell is a macrophage.

Preferred embodiments include methods wherein said antigen presenting cell is a monocyte.

Preferred embodiments include methods wherein said antigen presenting cell is an astrocyte.

Preferred embodiments include methods wherein said antigen presenting cell is a microglial cell.

Preferred embodiments include methods wherein said T helper cell possesses ability to license an antigen presenting cell to provide ability to activate a naïve T cell.

Preferred embodiments include methods wherein said T helper cell possesses ability to induce antigen presenting ability to a neutrophil.

Preferred embodiments include methods wherein said T helper cell possesses ability to induce antigen presenting ability to a basophil.

Preferred embodiments include methods wherein said T helper cell possesses ability to induce antigen presenting ability to an astrocyte.

Preferred embodiments include methods wherein said T helper cell possesses ability to induce antigen presenting ability to a telocyte.

Preferred embodiments include methods wherein said T helper cell possesses ability to induce B cell activation.

Preferred embodiments include methods wherein said B cell activation endows said B cell with ability to isotype switch.

Preferred embodiments include methods wherein said B cell activation endows said B cell with ability to differentiate into a plasma cell.

Preferred embodiments include methods wherein said B cell activation endows said B cell with ability activate NKT cells.

Preferred embodiments include methods wherein said B cell activation endows said B cell with ability to produce interleukin-10.

Preferred embodiments include methods wherein said B cell activation endows said B cell with ability to express FoxP3.

Preferred embodiments include methods wherein said B cell activation endows said B cell with ability to produce TGF-beta.

Preferred embodiments include methods wherein said B cell activation endows said B cell with ability to produce interleukin-35.

Preferred embodiments include methods wherein said IL-35 induces other B cells to differentiate into a B1 phenotype.

Preferred embodiments include methods wherein said B1 cells express CD5.

Preferred embodiments include methods wherein said B1 cells express CD10.

Preferred embodiments include methods wherein said B1 cells produce autocrine interleukin-10.

Preferred embodiments include methods wherein said T helper cell is capable of increasing activation state of natural killer cells.

The method claim 187, wherein said natural killer cell activation means increased ability of said natural killer cell to kill target cells.

The method claim 188, wherein said target cells are neoplastically transformed cells.

Preferred embodiments include methods wherein said neoplastically transformed cells are cells over expressing ras.

Preferred embodiments include methods wherein said neoplastically transformed cells are cells over expressing myc.

Preferred embodiments include methods wherein said neoplastically transformed cells are cells over expressing her2.

Preferred embodiments include methods wherein said neoplastically transformed cells are cells over expressing her2neu.

Preferred embodiments include methods wherein said neoplastically transformed cells are cells over expressing endosialin.

Preferred embodiments include methods wherein said neoplastically transformed cells are cells over expressing ROBO.

Preferred embodiments include methods wherein said neoplastically transformed cells are cells over expressing ROBO-4.

Preferred embodiments include methods wherein said neoplastically transformed cells are cells over expressing endoglin.

Preferred embodiments include methods wherein said neoplastically transformed cells are cells over expressing fibroblast growth factor 2.

Preferred embodiments include methods wherein said neoplastically transformed cells are cells over expressing fibroblast growth factor 1.

Preferred embodiments include methods wherein said neoplastically transformed cells are cells over expressing nanog.

Preferred embodiments include methods wherein said neoplastically transformed cells are cells over expressing OCT4.

Preferred embodiments include methods wherein said neoplastically transformed cells are cells over expressing kras.

Preferred embodiments include methods wherein said neoplastically transformed cells are cells over expressing epidermal growth factor receptor.

Preferred embodiments include methods wherein said neoplastically transformed cells are cells over expressing OCT4.

Preferred embodiments include methods wherein said target cells are cells which have been exposed to stressors.

Preferred embodiments include methods wherein said stressor is hyperthermia.

Preferred embodiments include methods wherein said stressor is hypotonicity.

Preferred embodiments include methods wherein said stressor is hypertonicity.

Preferred embodiments include methods wherein said stressor is hypothermia.

Preferred embodiments include methods wherein said hyperthermia is associated with induction of gp96.

Preferred embodiments include methods wherein said hyperthermia is associated with induction of hsp35.

Preferred embodiments include methods wherein said hyperthermia is associated with induction of hsp55.

Preferred embodiments include methods wherein said hyperthermia is associated with induction of hsp75.

Preferred embodiments include methods wherein said hyperthermia is associated with induction of hsp90.

Preferred embodiments include methods wherein said stressor is viral infection.

Preferred embodiments include methods wherein said stressor is bacterial infection.

Preferred embodiments include methods wherein said stressor is prion infection.

Preferred embodiments include methods wherein said stressor is endoplasmic reticulum stress.

Preferred embodiments include methods wherein said stressor is associated with proteosome deficiency.

Preferred embodiments include methods wherein said stressor is interferon response.

Preferred embodiments include methods wherein said interferon response is associated with activation of PKR.

Preferred embodiments include methods wherein said interferon response is associated with activation of RNAse H.

Preferred embodiments include methods wherein said interferon response is associated with activation of DNA repair responses.

Preferred embodiments include methods wherein said interferon response is associated with augmented levels of MICA.

Preferred embodiments include methods wherein said interferon response is associated with augmented levels of MICB.

Preferred embodiments include methods wherein said interferon response is associated with augmented levels of RoR-gamma.

Preferred embodiments include methods wherein said interferon response is associated with augmented levels of Rae-1.

Preferred embodiments include methods wherein said T regulatory cell activity is stimulatory of neurogenesis.

Preferred embodiments include methods wherein said T regulatory cell reduces age associated loss of neurogenesis.

Preferred embodiments include methods wherein said neurogenesis occurs in the dentate gyrus.

Preferred embodiments include methods wherein said neurogenesis occurs in the subventricular zone.

Preferred embodiments include methods wherein said neurogenesis occurs in the olfactory bulb.

Preferred embodiments include methods wherein said T regulatory cell reduces cerebral infarct associated loss of neurogenesis.

Preferred embodiments include methods wherein said T regulatory cell reduces depression associated loss of neurogenesis.

Preferred embodiments include methods wherein said T regulatory cell reduces cancer associated loss of neurogenesis.

Preferred embodiments include methods wherein said T regulatory cell reduces hepatic encephalopathy associated loss of neurogenesis.

Preferred embodiments include methods wherein said T regulatory cell reduces PTSD associated loss of neurogenesis.

Preferred embodiments include methods wherein said T regulatory cell reduces chronic traumatic encephalopathy associated loss of neurogenesis.

Preferred embodiments include methods wherein said T regulatory cell reduces surgery associated loss of neurogenesis.

Preferred embodiments include methods wherein said T regulatory cell reduces chemotherapy associated loss of neurogenesis.

Preferred embodiments include methods wherein said T regulatory cell reduces virus associated loss of neurogenesis.

Preferred embodiments include methods wherein said T regulatory cell reduces COVID-19 associated loss of neurogenesis.

Preferred embodiments include methods wherein said T regulatory cell reduces traumatic brain injury associated loss of neurogenesis.

Preferred embodiments include methods wherein said T regulatory cell reduces brain inflammation associated loss of neurogenesis.

Preferred embodiments include methods wherein said T regulatory cell reduces radiation associated loss of neurogenesis.

Preferred embodiments include methods wherein said T regulatory cell activity is stimulatory of angiogenesis.

Preferred embodiments include methods wherein said angiogenesis is associated with enhanced migration of endothelial cells towards an area of ischemia.

Preferred embodiments include methods wherein said ischemia is associated with activation of hypoxia inducible factor-1.

Preferred embodiments include methods wherein said activation of hypoxia inducible factor-1 results in enhanced production of VEGF.

Preferred embodiments include methods wherein said activation of hypoxia inducible factor-1 results in enhanced production of IGF-1.

Preferred embodiments include methods wherein said activation of hypoxia inducible factor-1 results in enhanced production of stromal derived factor-1.

Preferred embodiments include methods wherein said activation of hypoxia inducible factor-1 results in increased migration of angiogenesis promoting cells to the area of ischemia.

Preferred embodiments include methods wherein said angiogenesis promoting cell is monocytes.

Preferred embodiments include methods wherein said monocytes possess enhanced ability to secrete angiopoietin as compared to circulating monocytes in areas of normoxia.

Preferred embodiments include methods wherein angiogenesis promoting cells are neutrophils.

Preferred embodiments include methods wherein angiogenesis promoting cells are pericytes.

Preferred embodiments include methods wherein angiogenesis promoting cells are endothelial cells.

Preferred embodiments include methods wherein angiogenesis promoting cells are CD133 expressing endothelial cells.

Preferred embodiments include methods wherein angiogenesis promoting cells are CD34 expressing endothelial cells.

Preferred embodiments include methods wherein angiogenesis promoting cells are endothelial progenitor cells.

Preferred embodiments include methods wherein said endothelial progenitor cells are capable of differentiating into colony forming units expressing LDL receptor.

Preferred embodiments include methods wherein said endothelial progenitor cells are capable of binding to a lectin.

Preferred embodiments include methods wherein said lectin is concanavalin-A.

Preferred embodiments include methods wherein said lectin is phytohemagglutinin.

Preferred embodiments include methods wherein said lectin is pokeweed mitogen.

Preferred embodiments include methods wherein said lectin is CNA.

Preferred embodiments include methods wherein said lectin is concanavalin-A.

Preferred embodiments include methods wherein said endothelial progenitor cells express alpha v beta 3 integrin.

Preferred embodiments include methods wherein said endothelial progenitor cells express c-kit.

Preferred embodiments include methods wherein said endothelial progenitor cells express kdr.

Preferred embodiments include methods wherein said endothelial progenitor cells express MDA-5.

Preferred embodiments include methods wherein said endothelial progenitor cells express NOD-1.

Preferred embodiments include methods wherein said endothelial progenitor cells express alpha v beta 3 integrin.

Preferred embodiments include methods wherein said T regulatory cell activity is ability of said T regulatory cells to produce exosomes.

Preferred embodiments include methods wherein said T regulatory cell derived exosomes are between 30-250 nm in size.

Preferred embodiments include methods wherein said T regulatory cell derived exosomes are between 50-250 nm in size.

Preferred embodiments include methods wherein said T regulatory cell derived exosomes are between 75-200 nm in size.

Preferred embodiments include methods wherein said T regulatory cell derived exosomes express surface bound transforming growth factor beta.

Preferred embodiments include methods wherein said T regulatory cell derived exosomes express latency associated protein.

Preferred embodiments include methods wherein said T regulatory cell derived exosomes express miRNA-155

Preferred embodiments include methods wherein said T regulatory cell derived exosomes express tetraspanin.

Preferred embodiments include methods wherein said T regulatory cell derived exosomes express CD8.

Preferred embodiments include methods wherein said T regulatory cell derived exosomes express enhanced membrane glycosylation as compared to other cellular fragments.

Preferred embodiments include methods wherein said T regulatory cell derived exosomes express CD56.

Preferred embodiments include methods wherein said T regulatory cell derived exosomes express CD73.

Preferred embodiments include methods wherein said T regulatory cell derived exosomes express decay accelerating factor.

Preferred embodiments include methods wherein said T regulatory cell derived exosomes express membrane bound vimentin.

Preferred embodiments include methods wherein said T regulatory cell derived exosomes express membrane bound indolamine 2,3 dioxygenase.

Preferred embodiments include methods wherein said T regulatory cell derived exosomes suppress ability of dendritic cells to undergo maturation.

Preferred embodiments include methods wherein said T regulatory cell derived exosomes suppress ability of dendritic cells to undergo maturation in a TGF-beta dependent manner.

Preferred embodiments include methods wherein said T regulatory cell derived exosomes suppress ability of dendritic cells to undergo maturation in an interleukin-10 dependent manner.

Preferred embodiments include methods wherein said T regulatory cell derived exosomes suppress ability of dendritic cells to undergo maturation in a leukemia inhibitory factor dependent manner.

Preferred embodiments include methods wherein said T regulatory cell derived exosomes suppress ability of dendritic cells to undergo maturation in an HLA-E dependent manner.

Preferred embodiments include methods wherein said T regulatory cell derived exosomes suppress ability of dendritic cells to undergo maturation in an HLA-G dependent manner.

Preferred embodiments include methods wherein said T regulatory cell derived exosomes suppress ability of T helper cells to activate NF-AT.

Preferred embodiments include methods wherein said T regulatory cell derived exosomes suppress ability of T helper cells to produce interleukin-7.

Preferred embodiments include methods wherein said T regulatory cell derived exosomes suppress ability of T helper cells to produce interleukin-15.

Preferred embodiments include methods wherein said T regulatory cell derived exosomes suppress ability of T helper cells to proliferate in response to antigenic stimulation.

Preferred embodiments include methods wherein said T regulatory cell derived exosomes suppress ability of T helper cells to proliferate in response to TCR zeta chain phosphorylation.

Preferred embodiments include methods wherein said T regulatory cell derived exosomes suppress ability of T helper cells to proliferate in response to CD3 crosslinking.

Preferred embodiments include methods wherein said T regulatory cell derived exosomes suppress ability of T helper cells to proliferate in response to CD3 and CD28 crosslinking.

Preferred embodiments include methods wherein said T regulatory cell derived exosomes suppress ability of T helper cells to support maturation of antigen presenting cells in response to CD3 and CD28 crosslinking.

Preferred embodiments include methods wherein said antigen presenting cell is a dendritic cell.

Preferred embodiments include methods wherein said antigen presenting cell is a myeloid lineage.

Preferred embodiments include methods wherein said antigen presenting cell is a B cell.

Preferred embodiments include methods wherein said T regulatory cell activity is suppression of neuroinflammation.

Preferred embodiments include methods wherein said neuroinflammation is activation of microglial cells.

The method of claim 303, wherein said activated microglial produce an increase amount of TNF-alpha as compared to an unactivated microglial cell.

Preferred embodiments include methods wherein said activated microglial produce an increase amount of IL-1 beta as compared to an unactivated microglial cell.

Preferred embodiments include methods wherein said activated microglial produce an increase amount of IL-6 as compared to an unactivated microglial cell.

Preferred embodiments include methods wherein said activated microglial produce an increase amount of IL-18 as compared to an unactivated microglial cell.

Preferred embodiments include methods wherein said activated microglial produce an increase amount of IL-27 as compared to an unactivated microglial cell.

Preferred embodiments include methods wherein said activated microglial produce an increase amount of complement C5a as compared to an unactivated microglial cell.

Preferred embodiments include methods wherein said activated microglial produce an increase amount of complement C3a as compared to an unactivated microglial cell.

Preferred embodiments include methods wherein said activated microglial produce an increase amount of indolamine 2,3 dioxygenase as compared to an unactivated microglial cell.

Preferred embodiments include methods wherein said activated microglial produce an increase amount of TRANCE as compared to an unactivated microglial cell.

Preferred embodiments include methods wherein said activated microglial produce an increase amount of TRAIL as compared to an unactivated microglial cell.

Preferred embodiments include methods wherein said neuroinflammation is associated with enhance levels of NMDA receptor activation.

Preferred embodiments include methods wherein said neuroinflammation is associated with enhance levels glutaminergic signaling.

Preferred embodiments include methods wherein said neuroinflammation is associated with reduced levels of T regulatory cells in the brain environment.

Preferred embodiments include methods wherein said neuroinflammation is associated with Th17 cell activation in the central nervous system.

Preferred embodiments include methods wherein said Th17 cell activation induces microglial expression of HLA-DR.

Preferred embodiments include methods wherein said Th17 cell activation induces microglial expression of CD80.

Preferred embodiments include methods wherein said Th17 cell activation induces microglial expression of CD40.

Preferred embodiments include methods wherein said Th17 cell activation induces microglial expression of CD86.

Preferred embodiments include methods wherein said Th17 cell activation reduces microglial expression of HLA-G.

Preferred embodiments include methods wherein said Th17 cell activation induces endothelial expression of VCAM-1.

Preferred embodiments include methods wherein said Th17 cell activation induces endothelial expression of nitric oxide.

Preferred embodiments include methods wherein said Th17 cell activation induces endothelial expression NF-kappa B.

Preferred embodiments include methods wherein said Th17 cell activation induces endothelial expression of STAT3.

Preferred embodiments include methods wherein said Th17 cell activation induces endothelial expression of i-kappa B.

Preferred embodiments include methods wherein said Th17 cell activation increases ability of endothelium to allow for adhesion of inflammatory cells.

Preferred embodiments include methods wherein said inflammatory cells are CD4 T cells.

Preferred embodiments include methods wherein said CD4 T cells are Th1 cells.

Preferred embodiments include methods wherein said CD4 T cells are Th17 cells.

Preferred embodiments include methods wherein said inflammatory cells are neutrophils.

Preferred embodiments include methods wherein said neutrophils are capable of performing antigen presentation.

Preferred embodiments include methods wherein said neutrophils are capable of producing defesin B.

Preferred embodiments include methods wherein said neutrophils are N1 neutrophils.

Preferred embodiments include methods wherein said inflammatory cells are monocytes.

Preferred embodiments include methods wherein said monocytes are M1 monocytes.

Preferred embodiments include methods wherein said inflammatory cells are NK cells.

Preferred embodiments include methods wherein said inflammatory cells are NKT cells.

Preferred embodiments include methods wherein said inflammatory cells are gamma delta T cells.

Preferred embodiments include methods wherein said inflammatory cells are mast cells.

Preferred embodiments include methods wherein said inflammatory cells are telocytes.

Preferred embodiments include methods wherein said inflammatory cells are basophils.

Preferred embodiments include methods wherein said inflammatory cells are eosinophils.

Preferred embodiments include methods wherein said neuroinflammation is characterized by decreased neurogenesis.

Preferred embodiments include methods wherein said decreased neurogenesis is observed in the dentate gyrus.

Preferred embodiments include methods wherein said decreased neurogenesis is observed in the subventricular zone.

Preferred embodiments include methods wherein said decreased neurogenesis is associated with reduction of proliferation of CD133 expressing cells in the central nervous system.

Preferred embodiments include methods wherein said decreased neurogenesis is associated with reduction of proliferation of c-met expressing cells in the central nervous system.

Preferred embodiments include methods wherein said decreased neurogenesis is associated with reduction of proliferation of interleukin-3 receptor expressing cells in the central nervous system.

Preferred embodiments include methods wherein said decreased neurogenesis is associated with reduction of proliferation of BDNF receptor expressing cells in the central nervous system.

Preferred embodiments include methods wherein said decreased neurogenesis is associated with reduction of proliferation of NGF receptor expressing cells in the central nervous system.

Preferred embodiments include methods wherein said decreased neurogenesis is associated with reduction of proliferation of interleukin-6 receptor expressing cells in the central nervous system.

Preferred embodiments include methods wherein said T regulatory cell activity stimulate endogenous progenitor cell activation.

Preferred embodiments include methods wherein said T regulatory cells protect endogenous progenitor cells from inflammation associated inhibition.

Preferred embodiments include methods wherein said inflammatory stimuli is mediators released after cellular necrosis.

Preferred embodiments include methods wherein said inflammatory stimuli is mediators released after cellular pyroptosis.

Preferred embodiments include methods wherein said inflammatory stimuli is mediators released after cellular ferroptosis.

Preferred embodiments include methods wherein said inflammatory stimuli is mediators released after cellular apoptosis.

Preferred embodiments include methods wherein said inflammatory stimuli is HMGB1.

Preferred embodiments include methods wherein said inflammatory stimuli is TNF-alpha.

Preferred embodiments include methods wherein said inflammatory stimuli is IL-1 beta.

Preferred embodiments include methods wherein said inflammatory stimuli is tissue factor.

Preferred embodiments include methods wherein said inflammatory stimuli is a complement component

Preferred embodiments include methods wherein said inflammatory stimuli is small molecular weight hyaluronic acid fragments.

Preferred embodiments include methods wherein said inflammatory stimuli is a toll like receptor agonist.

Preferred embodiments include methods wherein said inflammatory stimuli is double stranded RNA.

Preferred embodiments include methods wherein said inflammatory stimuli is free DNA.

Preferred embodiments include methods wherein said inflammatory stimuli is circular DNA.

Preferred embodiments include methods wherein said inflammatory stimuli is double stranded RNA.

Preferred embodiments include methods wherein said inflammatory stimuli is lipopolysaccharide.

Preferred embodiments include methods wherein said inflammatory stimuli is flagellin.

Preferred embodiments include methods wherein said inflammatory stimuli is uric acid crystals.

Preferred embodiments include methods wherein said inflammatory stimuli is interferon alpha.

Preferred embodiments include methods wherein said inflammatory stimuli is interferon beta.

Preferred embodiments include methods wherein said inflammatory stimuli is interferon gamma.

Preferred embodiments include methods wherein said inflammatory stimuli is interferon lambda.

Preferred embodiments include methods wherein said inflammatory stimuli is interferon tau.

Preferred embodiments include methods wherein said decrease in regenerative activity of endogenous progenitor cells is characterized by increased fibrosis.

Preferred embodiments include methods wherein said decrease in regenerative activity of endogenous progenitor cells is characterized by increased collagen deposition.

Preferred embodiments include methods wherein said decrease in endogenous progenitor cells is characterized by increased tissue inhibitor of matrix metalloproteases.

Preferred embodiments include methods wherein said decrease in endogenous progenitor cells is characterized by decreased plasminogen activity.

Preferred embodiments include methods wherein said decrease in endogenous progenitor cells is characterized by decreased MMP1 activity.

Preferred embodiments include methods wherein said decrease in endogenous progenitor cells is characterized by decreased MMP3 activity.

Preferred embodiments include methods wherein said decrease in endogenous progenitor cells is characterized by decreased MMP5 activity.

Preferred embodiments include methods wherein said decrease in endogenous progenitor cells is characterized by decreased MMP9 activity.

Preferred embodiments include methods wherein said decrease endogenous progenitor cells is characterized by decreased MMP12 activity.

Preferred embodiments include methods wherein said decrease in endogenous progenitor cells is characterized by decreased TGF-alpha activity.

Preferred embodiments include methods wherein said decrease in endogenous progenitor cells is characterized by decreased TGF-beta activity.

Preferred embodiments include methods wherein said T regulatory cell activity decreases permeability of the blood brain barrier.

Preferred embodiments include methods wherein said decrease in permeability of said blood brain barrier allows for suppressed infiltration of inflammatory cells into the central nervous system.

Preferred embodiments include methods wherein said decreased permeability of said blood brain barrier allows for increased trafficking of T regulatory cells into the central nervous system.

Preferred embodiments include methods wherein said T regulatory cells express membrane bound TGF-beta.

Preferred embodiments include methods wherein said T regulatory cells express CTLA-4.

Preferred embodiments include methods wherein said T regulatory cells express membrane bound fas ligand.

Preferred embodiments include methods wherein said T regulatory cells express GITR.

Preferred embodiments include methods wherein said T regulatory cells express FoxP3.

Preferred embodiments include methods wherein said T regulatory cells secrete IL-10.

Preferred embodiments include methods list of compounds possessing pharmacologically acceptable properties for therapeutic use are derived from a drug library.

Preferred embodiments include methods wherein said drug library contains FDA approved compounds.

Preferred embodiments include methods wherein said drug library is the Selleckchem compound library.

Preferred embodiments include methods wherein said drug library is the MedChem Express compound library.

Preferred embodiments include methods wherein said drug library is the DiscoveryProbe compound library.

Preferred embodiments include methods wherein a library of compounds is utilized which possesses significant variability and chemical diversity.

Preferred embodiments include methods wherein said library is Enamine's Screening Collection.

Preferred embodiments include methods wherein said artificial intelligence means is a neural network.

Preferred embodiments include methods wherein said neural network is a plurality of networks, wherein a first neural network module and a second neural network module are provided.

Preferred embodiments include methods wherein the first neural network module and the second neural network module each have a plurality of endpoints, wherein each of the endpoints comprise one or more inputs or one or more outputs.

Preferred embodiments include methods wherein said endpoints are T regulatory cell mediated activities.

Preferred embodiments include methods wherein a first endpoint of the endpoints of the first neural network module matches those of a second endpoint of the endpoints of the second neural network module; further comprising one or more processors; memory operably coupled with one or more of the processors, wherein the memory stores instructions that are executable by one or more of the processors to cause one or more of the processors to: generate a combined neural network model that combines at least the first neural network module and the second neural network module, wherein generating the combined neural network model comprises joining the first endpoint of the first neural network module and the second endpoint of the second neural network module; identify a first version identifier based on a previously stored mapping, in the one or more computer readable media, of the first version identifier to at least the first endpoint of the first neural network module, wherein the previously stored mapping comprises data defining an association, and wherein the first version identifier reflects that the first neural network module has been trained; identify a second version identifier based on an additional previously stored mapping, in the one or more computer readable media, of the second version identifier to at least the second endpoint of the second neural network module, wherein the additional previously stored mapping comprises data defining an additional association, and wherein the second version identifier reflects that the second neural network module has been trained; determine whether the first version identifier assigned to the first endpoint of the first neural network module matches the second version identifier assigned to the second endpoint of the second neural network module; in response to determining that the first version identifier does match the second version identifier: use the combined neural network model; in response to determining that the first version identifier does not match the second version identifier: train at least the first neural network module to generate a refined version of the combined neural network model, wherein training the first neural network module comprises applying training examples to the combined neural network model, and use the refined version of the combined neural network model.

Preferred embodiments include methods wherein said a deep learning means is used to develop digital representations of molecular shapes associated with upregulation of T regulatory cell activity.

Preferred embodiments include methods wherein said digital representations are optimized through medicinal chemistry means to generate therapeutic candidates.

Preferred embodiments include methods wherein said therapeutic candidates are screened with T regulatory cells and assessed for suppressive activity.

Preferred embodiments include methods wherein said screening is performed by assessment using a FoxP3 reporter assay.

Preferred embodiments include methods wherein said FoxP3 reporter assay involves placing a color emitting signal in front of FoxP3 binding sites in a manner in which activation of FoxP3 results in creation of a marker that can be used to select cells in which FoxP3 has been activated.

Preferred embodiments include methods wherein said assessment of said FoxP3 activation is performed using a T regulatory cell line.

Preferred embodiments include methods wherein said T regulatory cell line is created by transfecting T regulatory cell progenitors with one or more oncogenes.

Preferred embodiments include methods wherein said oncogene is a temperature sensitive large T antigen.

Preferred embodiments include methods wherein assessment of T regulatory cell activity is performed using primary T regulatory cells.

Preferred embodiments include methods wherein said primary T regulatory cells express CD4.

Preferred embodiments include methods wherein said primary T regulatory cells express CTLA4.

Preferred embodiments include methods wherein said primary T regulatory cells express CD28.

Preferred embodiments include methods wherein said primary T regulatory cells express GITR ligand.

A method of identifying drugs which modulate T regulatory cell activity, said system comprising: a computer system possesses a memory and a processor; further comprising programming instructions stored in the memory and operating on the processor, in which the first plurality of programming instructions, when operating on the processor, causes the computer system to: receive molecule training data comprising one or more representations of one or more chemical formulas, wherein said chemical formulas have various degrees of associated with modulation of T regulatory cell activity; furthermore enriching the molecule training data by querying data sources to find similar molecules or molecules with similar bioactivity; use the enriched molecule training data to train an encoder of a neural network, wherein the encoder determines where each molecule in the enriched molecule training data lies in a latent space; determine a subspace of the latent space comprising a candidate set of latent examples; sample points within the subspace and perform interpolations, perturbations, or both on the sample points to expand the candidate set of latent examples; and decode the candidate set of latent examples to reconstruct a candidate set of chemically valid molecules.

Preferred embodiments include methods wherein said training data is generated by assessment of compounds on T regulatory cell cultures for ability to augment angiogenic activity.

Preferred embodiments include methods wherein said training data is generated by assessment of compounds on T regulatory cell cultures for ability to augment neurogenic activity.

Preferred embodiments include methods wherein said training data is generated by assessment of compounds on T regulatory cell cultures for ability to augment anti-diabetic activity.

Preferred embodiments include methods wherein said training data is generated by assessment of compounds on T regulatory cell cultures for ability to augment hepatogenic activity.

Preferred embodiments include methods wherein said training data is generated by assessment of compounds on T regulatory cell cultures for ability to augment nephrogenic activity.

Preferred embodiments include methods wherein said training data is generated by assessment of compounds on T regulatory cell cultures for ability to augment regenerative activity.

Preferred embodiments include methods wherein said molecule training data is formatted in standard SMILES format.

Preferred embodiments include methods wherein said SMILES format is enumerated to include non-standard information.

Preferred embodiments include methods wherein during enrichment, the de novo module interpolates between two known molecules to compensate for data disparity in the molecule training data.

Preferred embodiments include methods further comprising a bioactivity module comprising a second plurality of programming instructions stored in the memory and operating on the processor, wherein the second plurality of programming instructions, when operating on the processor, causes the computer system to rank the molecules in the candidate set of chemically valid molecules against target receptors.

Preferred embodiments include methods further comprising a reinforcement learning component that provides an additional gradient signal used to check chemical validity of decoded molecules.

Preferred embodiments include methods in which a reward prediction network for predicting the validity of an input graph.

A method of identifying drugs capable of modulating T regulatory cell activity, wherein said method consists of utilizing: a) a non-transitory computer-readable memory; and b) a processor configured to execute instructions stored on the non-transitory computer-readable memory which, when executed, cause the processor to identify a set of compounds based on one or more of a defined T regulatory cell activities, a set of desired characteristics, and a defined class of compounds, wherein said system pre-processes each compound of the set of compounds to generate respective sets of feature data;

process the sets of feature data with one or more trained machine learning models to produce predicted characteristic values for each compound of the set of compounds for each of the set of desired characteristics, wherein the one or more trained machine learning models are selected based on at least the set of desired characteristics, wherein the sets of feature data comprise a first set of feature data comprising one or more element interactive curvatures.

Preferred embodiments include methods wherein said feature data is ability of compounds to enhance T regulatory cell expression of FoxP3.

Preferred embodiments include methods wherein said feature data is ability of compounds to enhance T regulatory cell expression of TGF-beta.

Preferred embodiments include methods wherein said feature data is ability of compounds to enhance T regulatory cell expression of interleukin-10.

Preferred embodiments include methods wherein said feature data is ability of compounds to enhance T regulatory cell expression of interleukin-1 receptor antagonist.

Preferred embodiments include methods wherein said feature data is ability of compounds to enhance T regulatory cell expression of soluble HLA-G.

Preferred embodiments include methods wherein said feature data is ability of compounds to enhance T regulatory cell expression of Fas ligand.

Preferred embodiments include methods wherein said feature data is ability of compounds to enhance T regulatory cell ability to suppress proliferation of a conventional T cell.

Preferred embodiments include methods wherein said conventional T cell is stimulated to proliferate by phosphorylation of T cell receptor ITAMS.

Preferred embodiments include methods wherein said conventional T cell is stimulated to proliferate by dephosphorylation of T cell receptor ITAMS.

Preferred embodiments include methods wherein said conventional T cell is stimulated to proliferate by T cell receptor mediated activation.

Preferred embodiments include methods wherein said conventional T cell is stimulated to proliferate by ligation of CD3.

Preferred embodiments include methods wherein said conventional T cell is stimulated to proliferate by ligation of CD3 and CD28.

Preferred embodiments include methods wherein said conventional T cell is stimulated to proliferate by treatment with interleukin-2.

Preferred embodiments include methods wherein said conventional T cell is stimulated to proliferate by treatment with interleukin-7.

Preferred embodiments include methods wherein said conventional T cell is stimulated to proliferate by treatment with interleukin-15.

Preferred embodiments include methods wherein said conventional T cell is stimulated to proliferate by treatment with a mitogen.

Preferred embodiments include methods wherein the instructions, when executed, cause the processor, or the system to: assign rankings to each compound of the set of compounds for each characteristic of the set of desired characteristics, wherein assigning a ranking to a given compound of the set of compounds for a given characteristic of the set of desired characteristics comprises: comparing a first predicted characteristic value of the predicted characteristic values corresponding to the given compound to other predicted characteristic values of other compounds of the set of compounds, wherein the ordered list is ordered according to the assigned rankings.

Preferred embodiments include methods wherein the set of compounds includes protein-ligand complexes, especially FoxP3, and wherein the instructions, when executed, further cause the processor to, for a first protein-ligand complex of the protein-ligand complexes: determine an element interactive density for the first protein-ligand complex; identify a family of interactive manifolds for the first protein-ligand complex; determine an element interactive curvature based on the element interactive density; and generate a set of feature vectors based on the element interactive curvature, wherein the first set of feature data includes the set of feature vectors, wherein the one or more element interactive curvatures comprise the element interactive curvature, wherein the set of desired characteristics comprises protein binding affinity, wherein the one or more trained machine learning models comprise a machine learning model that is trained to predict protein binding affinity values based on the set of feature vectors, and wherein the predicted characteristic values comprise the predicted protein binding affinity values.

Preferred embodiments include methods wherein the instructions, when executed, further cause the processor to: determine an element interactive density for a first compound of the set of compounds; identify a family of interactive manifolds for the first compound; determine an element interactive curvature based on the element interactive density; and generate a set of feature vectors based on the element interactive curvature, wherein the first set of feature data includes the set of feature vectors, wherein the one or more element interactive curvatures comprise the element interactive curvature, wherein the set of desired characteristics comprises one or more toxicity endpoints, wherein the one or more trained machine learning models comprise a machine learning model that is trained to output predicted toxicity endpoints values corresponding to the one or more toxicity endpoints based on the set of feature vectors, and wherein the predicted characteristic values comprise the predicted toxicity endpoint values.

Preferred embodiments include methods wherein the instructions, when executed, further cause the processor to: determine an element interactive density for a first compound of the set of compounds; identify a family of interactive manifolds for the first compound; determine an element interactive curvature based on the element interactive density; and generate a set of feature vectors based on the element interactive curvature, wherein the one or more element interactive curvatures comprise the element interactive curvature, wherein the first set of feature data includes the set of feature vectors, wherein the set of desired characteristics comprises solvation free energy, wherein the one or more trained machine learning models comprise a machine learning model that is trained to output predicted solvation free energy values corresponding to a solvation free energy of the first compound based on the set of feature vectors, and wherein the predicted characteristic values comprise the predicted solvation free energy values.

### DETAILED DESCRIPTION OF THE INVENTION

At least one specification heading is required. Please delete this heading section if it is not applicable to your application. For more information regarding the headings of the specification, please see MPEP 608.01(a).

The invention discloses the utilization of artificial intelligence systems and/or fuzzy logic based systems for identification of compounds capable of modulating T regulatory cell activity.

“Bioactivity” as used herein means the physiological effects of a molecule on an organism.

“Edges” as used herein means connections between nodes or vertices in a data structure. In graphs, an arbitrary number of edges may be assigned to any node or vertex, each edge representing a relationship to itself or any other node or vertex. Edges may also comprise value, conditions, or other information, such as edge weights or probabilities.

“FASTA” as used herein means any version of the FASTA family (e.g., FASTA, FASTP, FASTA, etc.) of chemical notations for describing nucleotide sequences or amino acid (protein) sequences using text (e.g., ASCII) strings.

“Ligand” as used herein means a substance that forms a complex with a biomolecule to serve a biological purpose. In protein-ligand binding, the ligand is usually a molecule which produces a signal by binding to a site on a target protein. Ligand binding to a receptor protein alters the conformation by affecting the three-dimensional shape orientation. The conformation of a receptor protein composes the functional state. Ligands comprise substrates, inhibitors, activators, signaling lipids, and neurotransmitters.

“Nodes” and “Vertices” are used herein interchangeably to mean a unit of a data structure comprising a value, condition, or other information. Nodes and vertices may be arranged in lists, trees, graphs, and other forms of data structures. In graphs, nodes and vertices may be connected to an arbitrary number of edges, which represent relationships between the nodes or vertices. As the context requires, the term “node” may also refer to a node of a neural network (also referred to as a neuron) which is analogous to a graph node in that it is a point of information connected to other points of information through edges.

“Proteins” as used herein means large biomolecules, or macromolecules, consisting of one or more long chains of amino acid residues. Proteins perform a vast array of functions within organisms, including catalyzing metabolic reactions, DNA replication, responding to stimuli, providing structure to cells and organisms, and transporting molecules from one location to another. Proteins differ from one another primarily in their sequence of amino acids, which is dictated by the nucleotide sequence of their genes, and which usually results in protein folding into a specific 3D structure that determines its activity.

“SMILES” as used herein means any version of the “simplified molecular-input line-entry system,” which is form of chemical notation for describing the structure of molecules using short text (e.g., ASCII) strings.

In one embodiment the invention provides systems and/or methodologies which embed deep learning into a rule-based technique powered by the fuzzy inference system. The novelty of this approach relies on the fusion of two classifiers: the type-2 fuzzy sets (T2FS) and a deep neural network (DNN), where linguistic inputs are translated into representations that generate feature labels for the DNN system. The DNN then drives the fuzzy inference block through adjustable fuzzy rules incorporated by (domain) expert knowledge acquired from the input data—required to explain the behavior of the fuzzy system. To deal with the highly error-prone nature of real-world datasets, we also incorporate a multidimensional scaling technique for the purpose of enhancing the datasets for precise modelling and prediction of T regulatory cell responses to compounds that are tested.

In one embodiment systems are provided in which a library of agents possessing favorable pharmacological properties for drug development are chosen. Through utilization of deep learning approaches, which are commercially available, said agents are screening in the literature for ability to manipulate T regulatory cell activities.

For the practice of the current invention, “Deep learning” refers to a subset of machine learning that uses neural networks to model and solve complex problems. It is a type of artificial intelligence that involves training a neural network to recognize patterns in large sets of data. Deep learning is capable of learning to recognize patterns, features, and relationships in data without being explicitly programmed to do so. The “deep” in deep learning refers to the fact that these neural networks consist of multiple layers, allowing them to learn increasingly abstract representations of the input data. This enables them to perform tasks such as image and speech recognition, natural language processing, and even playing games at a superhuman level. TensorFlow, PyTorch, and Keras are examples of deep learning frameworks.

In part the novelty of the invention relies on assessment of various T regulatory cell properties that are not commonly known are sought during literature searches and drug development approaches. Properties of T regulatory cells useful for training the system to search include:

Suppression of antigen presentation. One of the fundamental triggers of immunity is the ability of the dendritic cells to induce activation of naïve T cells. Dendritic cells capture antigens from the environment and process them into small peptides which are then presented to T cells in the context of major histocompatibility complex (MHC) molecules on their surface. Dendritic cells express co-stimulatory molecules such as CD80 and CD86, which engage with co-stimulatory receptors (such as CD28) on the surface of T cells. This interaction provides a second signal to the T cells, which is necessary for their activation. After capturing and presenting antigen, dendritic cells migrate from peripheral tissues to secondary lymphoid organs (such as lymph nodes) where they can encounter naive T cells. Upon encountering a dendritic cell presenting antigen and providing co-stimulation, naive T cells undergo clonal expansion, differentiation, and acquisition of effector functions, which ultimately leads to the activation of the adaptive immune response. By identifying agents from a chemical library associated with suppression of in vivo antigen presentation, it is possible to choose candidates for further testing.

Assays for assessment of antigen presenting activity maybe performed using numerous methodologies. The present invention relates in general to screening of T regulatory modulators by assessment of dendritic cells and their association with immune responses. The present invention provides methods of making dendritic cell gene expression profile libraries and the libraries made thereof in response to microbial stimuli. In addition, the present invention provides methods of inducing IL-2 in dendritic cells and using IL-2 to activate lymphocytes and immune responses in association with dendritic cells. The present invention also provides methods and systems useful for screening agents capable of affecting dendritic cell maturation. The present invention also provides methods for screening candidate therapeutic agents suitable for modulating T regulatory cell activation. According to the present invention, methods for making a gene expression profile library for dendritic cells exposed to a T regulatory cell stimulus include incubating immature dendritic cells with a T regulatory cell e.g., dendritic cell maturation stimulus, identifying genes in the dendritic cells that have changed their levels of expression in response to the T regulatory cell stimulus, e.g., either a substantial increase or decrease of the gene expression level, and generating a gene expression profile, e.g., in a computer readable media indicating the genes and levels of changes corresponding to the stimulus.

machine learning may be applied in biomolecular data analysis and prediction. In particular, deep neural networks may recognize nonlinear and high-order interactions among features as well as the capability of handling data with underlying spatial dimensions. Machine learning based approaches to data-driven discovery of structure-function relationships may be advantageous because of their ability to handle very large data sets and to account for nonlinear relationships in physically derived descriptors. For example, deep learning algorithms, such as deep convolutional neural networks may have the ability to automatically extract optimal features and discover intricate structures in large data sets, as will be described. However, the way that biological datasets are manipulated and organized before being presented to machine learning systems can provide important advantages in terms of performance of systems and methods that use trained machine learning to perform real world tasks. When there are multiple learning tasks, multi-task learning (MTL) may be applied as a powerful tool to, for example, exploit the intrinsic relatedness among learning tasks, transfer predictive information among tasks, and achieve better generalized performance. During the learning stage, MTL algorithms may seek to learn a shared representation (e.g., shared distribution of a given hyper-parameter, shared low-rank subspace shared feature subset and clustered task structure), and use the shared representation to bridge between tasks and transfer knowledge. MTL, for example, may be applied to identify bioactivity of small molecular drugs and genomics. Linear regression based MTL may depend on “well-crafted” features while neural network based MTL may allow more flexible task coupling and is able to deliver decent results with large number of low level features as long as such features have the representation power of the problem. For complex 3D biomolecular data, such as interaction between FoxP3 or other T regulatory cell associated proteins with the screened molecules, the physical features used as inputs to machine learning algorithms may vary greatly in their nature (e.g., depending on the application). Typical features may be generated from geometric properties, electrostatics, atomic type, atomic charge and graph theory properties, for example. Such extracted features can be fed to a deep neural network, for example. Performance of the deep neural network may be reliant on the fashion of feature construction. On the other hand, convolutional neural networks may be capable of learning high level representations from low level features. However, the cost (e.g., computational cost) of directly applying a convolutional neural network to 3D biomolecules may be considerable when long range interactions need to be considered. There is presently a need for a competitive deep learning algorithm for directly predicting protein-ligand binding affinities and protein folding stability changes upon mutation from 3D biomolecular data sets. Additionally, there is a need for a robust multi-task deep learning method for improving both protein-ligand (or protein-protein, or protein-nucleic acid) binding affinity and mutation impact predictions, as well as solvation, toxicity, and other characteristics.

Another approach, in another embodiment is the use of topology based approaches for the determination of structure-function relationships of biomolecules may provide a dramatic simplification to biomolecular data compared to conventional geometry based approaches. Generally, the study of topology deals with the connectivity of different components in a space, and characterizes independent entities, rings and higher dimensional faces within the space. Topological models may provide the best level of abstraction of many biological processes, such as the open or close state of ion channels, the assembly or disassembly of virus capsids, the folding and unfolding of proteins, and the association or dis-association of ligands (or proteins). A fundamental task of topological data analysis may be to extract topological invariants, namely the intrinsic features of the underlying space, of a given data set without additional structure information. For example, topological invariants may be embedded with covalent bonds, hydrogen bonds, van der Waals interactions, etc. A concept in algebraic topology methods is simplicial homology, which concerns the identification of topological invariants from a set of discrete node coordinates such as atomic coordinates in a protein or a protein-ligand complex. For a given (protein) configuration, independent components, rings and cavities are topological invariants and their numbers are called Betti-0, Betti-1 and Betti-2, respectively, as will be described. However, pure topology or homology may generally be free of metrics or coordinates, and thus may retain too little geometric information to be practically useful.

In one embodiment, gene expression profiles are generated by identifying differentially expressed genes in dendritic cells upon exposure to a microbial stimulus in the presence of T regulatory cells. The regulatory cell modulating agents are screened and the input is provided to the deep learning system. A microbial stimulus of the present invention can be any stimulation that triggers dendritic cell maturation, e.g., IL-2 production of dendritic cells. For example, a microbial stimulus can be a microorganism or one or more products or components thereof. In one embodiment, the microbial stimulus of the present invention includes microorganisms, e.g., bacteria, viruses, fungal organisms and prions. In another embodiment, the microbial stimulus of the present invention includes Gram+bacteria, lipoteichoic acid (LTA, a component of Gram+bacteria), Gram-bacteria, LPS (a component of Gram-bacteria), oligonucleotides containing unmethylated CpG motif, zymosan, yeasts, e.g., Saccaromycies Cerevisiae, and stimuli mediated by T cell help such as anti-CD40 antibodies. Levels of gene expressions can be determined using any suitable means available to one skilled in the art. For example, levels of gene expression can be determined by detecting the levels of gene transcripts using microarrays representing 11000 genes and expressed sequence tags (ESTs). One way of analyzing levels of gene expression in general is by using Principal Component Analysis (PCA) method, which allows the dimensionality of complex data to be reduced.] Differentially expressed genes can be identified using any means known to one skilled in the art. For example, a first gene clustering algorithm can be used which groups genes according to the similarity of their expression patterns based on Self-Organizing Maps (SOMs). Genes or ESTs are excluded from the profile if the changes of their expression are below a predetermined level based on mean average differences. Each SOMs can also be further analyzed using a second gene clustering method, e.g., Hierarchical clustering. According to another aspect of the invention, IL-2 production in dendritic cells is associated with activation of toll like receptors (TLRs) of dendritic cells or T cell help mediated stimulation to dendritic cells. Therefore, the present invention provides methods for inducing IL-2 production in dendritic cells by contacting dendritic cells with an agent activating one or more TLRs in dendritic cells or stimulating dendritic cells via T cell help. Such agent can be any known or later discovered agent including, without limitation, a microbial stimulus. In one embodiment, such agent does not include any inflammatory cytokines. TLRs activated by the agent can be any TLRs of dendritic cells including, without limitation, TLR2, TLR4, and TLR9. Dendritic cells obtained by such method can be used for any purpose either in vivo or in vitro. For example, dendritic cells containing activated TLRs can be used for cell-based therapies, e.g., inducing immune responses for therapeutic treatment of malignant growth or infectious diseases. According to another aspect of the invention, the present invention provides methods useful for screening agents capable of affecting dendritic cell activation or maturation. The method includes incubating in the presence and absence of a test agent, a microbial stimulus and immature dendritic cells, and detecting one or more activities that are specific to dendritic cell activation or maturation in the presence and absence of the test agent. An increase or decrease in the amount of the activities specific to dendritic cell activation or maturation caused by the test agent is indicative of an agent capable of affecting dendritic cell activation or maturation. The test agents used in the screening methods of the present invention can be any agent to be tested for therapeutic uses. In one embodiment, the test agents are compounds, small molecules, polynucleotides, polypeptides, and any derivatives thereof. Activities that are specific to dendritic cell activation or maturation include any activity associated specifically with dendritic cell activation or maturation. For example, several activities are specifically associated with dendritic cells upon their encountering of a microbial stimulus and these activities include, without limitation, antigen intake, production of cytokines, activation of lymphocytes such as priming naïve T cells, and expression of cell surface proteins such as MHC-I, MHC-II, CD40, CD54, CD80, and CD86. In one embodiment, IL-2 expression is used as one of the activities specific to dendritic cell activation and is detected in the presence and absence of a test agent. The present invention also provides an assay system useful for testing an agent's ability to affect dendritic cell maturation. The system includes a container containing a test agent, a microbial stimulus and immature dendritic cells. The system can include one or more containers and can be used directly or in connection with other systems to detect IL-2 expression of dendritic cells in the presence and absence of a test agent and/or collecting data in a computer readable medium. In one embodiment, the system is a high-throughput system. Activities specific for dendritic cell activation include any activity specifically associated with dendritic cells' response upon their encountering of a microbial stimulus. For example, these activities can include, without limitation, antigen intake, production of cytokines, activation of lymphocytes such as priming naïve T cells, response to microbial stimuli, and expression of cell surface proteins such as MHC-I, MHC-II, CD40, CD54, CD80, and CD86. In one embodiment, IL-2 expression is used as one of the activities specific to dendritic cell activation and is detected in the presence and absence of a test agent.

In one embodiment compounds are screened utilizing deep learning and natural language processing. In one embodiment, the invention teaches an architecture is specifically designed and structured into two major phases namely: (i) data collection and processing, and (ii) T regulatory response modelling and optimization. The modelling-optimization phase fusses a two-stage classification system with MDS capability, into a hybridized controller capable of high error-tolerant patient response modelling and optimization. The controller accepts through a fuzzy interface, linguistic inputs (parameters) from a processed database of unique experimental (Stanford and locally sourced) datasets. Supervised learning is then achieved through the automatic adjustment of the fuzzy model parameters which forms initial inputs to the DNN and initiated by the learning algorithm. An optimized set of non-fuzzy inputs are then fed into the IT2FL section to output precise patient response, which errors are later pruned using an MDS algorithm. The pruned datasets are finally learned to produce optimized predictions of the T regulatory cell responses. In some embodiments of the invention, systems and methods for processing unstructured information such as describing T regulatory cell manipulating substances are provided. In particular, these systems and methods provide automatic processing of text information in scientific lexicon or natural language. The methods may extract information from natural language texts, seek information in collections of documents, and/or monitor information. In some specific embodiments, the described systems and methods may provide a universal core independent of the specific language and a lexical content that includes a language-specific lexicon and language models for word formation and word change, as well as syntactic models for coordination and word use in that language. On the other hand, a universal language-independent core includes the exhaustive set of knowledge about the world and ways how the knowledge may be expressed in a language. The knowledge may be represented in the form of hierarchic description of entities of the world, their properties, possible attributes, their relationships, and ways to express it in a language. This type of semantic description may be useful for creating smart natural language processing (“NLP”) technologies, especially, applications which can “understand the sense” expressed in natural language, they are necessary to create applications and to solve many natural language processing tasks such as Machine Translation, Semantic Indexing, and Semantic Search, including Multilingual Semantic Search, Fact Extraction, Sentiment Analysis, Similar Document Search, Document Classification, Summarization, Big Data analysis, eDiscovery, Morphology & Lexical Analyzer, and similar applications. In specific embodiments, the disclosed systems and methods may store and operate on text units-words, sentences, texts-in the data base, and also do so with lexical and semantic meanings for the words, sentences, texts and other information units. Any thought, concept, notification, any fact or anything said in a language can be expressed using sentences. Every sentence is represented as a sequence of lexical meanings joined by certain relationships, which is expressed in the language as filling the surface (syntactic) slots, and at the semantic level the deep (semantic) slots. For example, in the sentence “The girl eats the apple,” the word “apple” fills the slot for Object of the verb “eat”, and “girl” fills its surface slot for Subject. The nomenclature for surface slots may be rather broad and differ in different languages. The differences are due to the difference in syntactic models in different languages. At the semantic level, the lexical meaning of “girl” fills the deep slot named “Agens”, while the lexical meaning of “apple” fills the deep slot “Object.” Through using similar methodologies the invention teaches the utilization of machine reading to search the literature, identify compounds from a library of which are associated with activities that suggest ability of compounds to alter T regulatory cell activity.

In some examples, neural learning systems, including deep learning, and stochastic based fuzzy logic systems are utilized to search pubchem and develop various structure-function relationship maps, which are further utilized as the basis for in silico and subsequently in vitro screening.

1. A method of identifying agents capable of modulating T regulatory cell activity comprising the steps of:
a) identifying a list of compounds possessing pharmacologically acceptable properties for therapeutic use;
b) utilizing an artificial intelligence system to assess ability of said compounds from step (a) to modulate ability of FoxP3 protein to interact with target sites on DNA;
c) enhancing ability of said compounds identified from step (b) to modulate FoxP3 DNA binding by performing chemical optimization steps; and
d) assessing activity of said identified compounds using in vitro T regulatory cell assessment assays.

2. The method of claim 1, wherein said T regulatory cell activity is magrophage activity and said macrophages are plastic adherent cells.

3. The method of claim 2, wherein said macrophages are capable of generating TNF-alpha after TLR4 activation.

4. The method of claim 2, wherein said macrophages increase expression of HLA-I upon treatment with interferon gamma.

5. The method of claim 2, wherein said macrophages increase expression of HLA-I upon treatment with an activator of NF-kappa B.

6. The method of claim 5, wherein said activator of NF-kappa B is conditioned media from interleukin 17 treated mesenchymal stem cells.

7. The method of claim 6, wherein said mesenchymal stem cells express HLA-G.

8. The method of claim 6, wherein said activator of NF-kappa B is conditioned media from interleukin 6 treated dendritic cells.

9. The method of claim 6, wherein said activator of NF-kappa B is conditioned media from interleukin 6 treated type 1 B cells.

10. The method of claim 2, wherein said T regulatory cells are assessed for ability to suppress complement C3 induced maturation of M1 macrophages.

11. The method of claim 2, wherein said T regulatory cell modulation of macrophage activity is upregulation of activity of M2 macrophages.

12. The method of claim 11, wherein said M2 macrophages are preferentially angiogenic as compared to M1 macrophages.

13. The method of claim 12, wherein said preferential angiogenic activity of M2 macrophages is due to increased production of angiogenin as compared to M1 macrophages.

14. The method of claim 12, wherein said preferential angiogenic activity of M2 macrophages is due to increased production of follistatin as compared to M1 macrophages.

15. A method of identifying drugs capable of modulating T regulatory cell activity, wherein said method consists of utilizing: a) a non-transitory computer-readable memory; and b) a processor configured to execute instructions stored on the non-transitory computer-readable memory which, when executed, cause the processor to identify a set of compounds based on one or more of a defined T regulatory cell activities, a set of desired characteristics, and a defined class of compounds, wherein said system pre-processes each compound of the set of compounds to generate respective sets of feature data; process the sets of feature data with one or more trained machine learning models to produce predicted characteristic values for each compound of the set of compounds for each of the set of desired characteristics, wherein the one or more trained machine learning models are selected based on at least the set of desired characteristics, wherein the sets of feature data comprise a first set of feature data comprising one or more element interactive curvatures.

16. The method of claim 15, wherein said feature data is the ability of compounds to enhance T regulatory cell expression of interleukin-10.

17. The method of claim 15, wherein the instructions, when executed, cause the processor, or the system to: assign rankings to each compound of the set of compounds for each characteristic of the set of desired characteristics, wherein assigning a ranking to a given compound of the set of compounds for a given characteristic of the set of desired characteristics comprises: comparing a first predicted characteristic value of the predicted characteristic values corresponding to the given compound to other predicted characteristic values of other compounds of the set of compounds, wherein the ordered list is ordered according to the assigned rankings.

18. The method of claim 15, wherein the set of compounds includes protein-ligand complexes, especially FoxP3, and wherein the instructions, when executed, further cause the processor to, for a first protein-ligand complex of the protein-ligand complexes: determine an element interactive density for the first protein-ligand complex; identify a family of interactive manifolds for the first protein-ligand complex; determine an element interactive curvature based on the element interactive density; and generate a set of feature vectors based on the element interactive curvature, wherein the first set of feature data includes the set of feature vectors, wherein the one or more element interactive curvatures comprise the element interactive curvature, wherein the set of desired characteristics comprises protein binding affinity, wherein the one or more trained machine learning models comprise a machine learning model that is trained to predict protein binding affinity values based on the set of feature vectors, and wherein the predicted characteristic values comprise the predicted protein binding affinity values.

19. The method of claim 15, wherein the instructions, when executed, further cause the processor to:
determine an element interactive density for a first compound of the set of compounds; identify a family of interactive manifolds for the first compound; determine an element interactive curvature based on the element interactive density; and generate a set of feature vectors based on the element interactive curvature, wherein the first set of feature data includes the set of feature vectors, wherein the one or more element interactive curvatures comprise the element interactive curvature, wherein the set of desired characteristics comprises one or more toxicity endpoints, wherein the one or more trained machine learning models comprise a machine learning model that is trained to output predicted toxicity endpoints values corresponding to the one or more toxicity endpoints based on the set of feature vectors, and wherein the predicted characteristic values comprise the predicted toxicity endpoint values.

20. The method of claim 15, wherein the instructions, when executed, further cause the processor to:
determine an element interactive density for a first compound of the set of compounds; identify a family of interactive manifolds for the first compound; determine an element interactive curvature based on the element interactive density; and generate a set of feature vectors based on the element interactive curvature, wherein the one or more element interactive curvatures comprise the element interactive curvature, wherein the first set of feature data includes the set of feature vectors, wherein the set of desired characteristics comprises solvation free energy, wherein the one or more trained machine learning models comprise a machine learning model that is trained to output predicted solvation free energy values corresponding to a solvation free energy of the first compound based on the set of feature vectors, and wherein the predicted characteristic values comprise the predicted solvation free energy values.
